(19) United States
(12) Patent Application Publication
(10) Pub. No.: US 2016/0262738 A1
(43) Pub. Date: Sep. 15, 2016

(54) SUPERELASTIC ARTICULATING MECHANISM

(71) Applicant: Nir Altman, Kfar Etzion (IL)

(72) Inventor: Nir Altman, Kfar Etzion (IL)

(21) Appl. No.: 14/645,614

(22) Filed: Mar. 12, 2015

(51) Int. Cl. A61B 17/00

(52) U.S. Cl. CPC A61B 17/00234; A61B 2017/00314; A61B 2017/00327; A61B 2017/00407

(57) ABSTRACT

A surgical device includes a handle, a drive shaft that protrudes from the handle, and an articulating applicator arm connected to the drive shaft. An articulation trigger includes at least one superelastic articulating cable that has one end connected to the articulation trigger and an opposite end connected to the articulating applicator arm.

### FIELD OF THE INVENTION

The present invention relates generally to surgical devices that have an articulating mechanism, such as but not limited to, a tacker for applying surgical fasteners, and particularly to a superelastic articulating mechanism.

### BACKGROUND OF THE INVENTION

A number of surgical, laparoscopic and endoscopic procedures require application of rotary tacks to tissues, such as for hernia repairs and the like.

Tackers for applying such rotary tacks are well known. A typical tacker drive and articulating mechanism of the prior art is shown in FIG. 1. A handle 1 houses a deployment trigger 2 for rotating a drive shaft 3 housed in an outer tube 3A. The deployment trigger 2 is spring-loaded by a spring 4. Squeezing the deployment trigger 2 (upwards, clockwise in the sense of the drawing) causes rotation of the drive shaft 3 through a gear train 5; the motion of the trigger causes a spur gear 6 to mesh with and turn a series of gears of the gear train 5 to cause rotation of the drive shaft 3. Rotary tacks disposed in an applicator arm (not shown) are rotatingly connected to the drive shaft 3. Operation of the deployment trigger 2 causes the drive shaft 3 to rotate so as to distally advance the rotary tacks from the applicator arm for deployment in tissue.

The tacker mechanism of the prior art also includes an articulation trigger 7 for articulating the applicator arm at different angles. Articulating cables 8 are connected to upper and lower points on articulation trigger 7 and enter the outer tube 3A through entry holes. The articulating cables 8 run through the length of outer tube 3A and are connected to an articulating portion (not shown) of the applicator arm. The articulation trigger 7 pivots about a pivot 9. Pulling the lower part of the articulation trigger 7 towards the handle 1 (upwards, counterclockwise in the sense of the drawing) puts tension on (that is, pulls) the lower of the articulating cables 8 and causes the applicator arm to articulate downwards. Conversely, pushing the lower part of the articulation trigger 7 away from the handle 1 (downwards, clockwise in the sense of the drawing) puts tension on (that is, pulls) the upper of the articulating cables 8 and causes the applicator arm to articulate upwards.

A problem can occur with the articulating cables 8 and articulation trigger 7. After using the articulation cables 8 to articulate the applicator arm, it is often desired to lock the trigger in place to fix the applicator arm at a particular orientation. The handle 1 is provided with a locking mechanism to lock the trigger 7 at a variety of places along the trigger movement. However, the articulating cables 8 are not designed to accurately place the trigger 7 at any of the locking positions; there are problems of free play and tolerances with the result that the trigger 7 does not automatically reach any of the locking positions.

In the prior art, the problem is solved with coil springs 11, which are connected to the upper and lower parts of the articulation trigger 7 and which provide tension on the trigger 7. The motion of the springs 11 and of articulation trigger 7 is constrained by a four-bar linkage mechanism 10, which ensures smooth, parallel movement of the springs 11 and trigger 7. If the trigger 7 were to misalign with a desired locking position, one of the springs is in tension and this pulls the trigger to the locking position.

However, the springs and four-bar linkage mechanism involve a relatively large amount of parts and high manufacturing and assembly costs.

### SUMMARY OF THE INVENTION

The present invention seeks to provide an improved articulating mechanism for surgical devices that require articulation, such as but not limited to, tackers, endoscopic devices, laparoscopic devices and others, as is described more in detail hereinbelow. In particular, the present invention seeks to provide a superelastic articulating mechanism that eliminates the drawbacks of the prior art; the number of parts is greatly reduced, and manufacturing and assembly costs are also significantly reduced.

There is thus provided in accordance with a non-limiting embodiment of the present invention a surgical device including a handle, a drive shaft that protrudes from the handle, an articulating applicator arm connected to the drive shaft, and an articulation trigger including at least one superelastic articulating cable that has one end connected to the articulation trigger and an opposite end connected to the articulating applicator arm.

In accordance with a non-limiting embodiment of the present invention the articulation trigger includes a locking mechanism for locking the articulation trigger at a selected operating position.

In accordance with a non-limiting embodiment of the present invention the handle further includes a deployment trigger for rotating the drive shaft.

In accordance with a non-limiting embodiment of the present invention, in a method for using the surgical device, the articulation trigger is operated so as to apply a tensioning force and elastically stretch the at least one superelastic articulating cable and cause articulation of the articulating applicator arm.

### BRIEF DESCRIPTION OF THE DRAWINGS

The present invention will be understood and appreciated more fully from the following detailed description taken in conjunction with the drawings in which:

FIG. 1 is a simplified pictorial illustration of an articulating mechanism of the prior art;

FIG. 2 is a simplified pictorial illustration of an articulating mechanism, constructed and operative in accordance with a non-limiting embodiment of the present invention;

FIG. 3 is a simplified pictorial illustration of a trigger of the articulating mechanism; and

FIG. 4 is a simplified pictorial illustration of the trigger of the articulating mechanism at one end of a drive shaft in a tube and an articulating applicator arm at the other end of the tube, in accordance with a non-limiting embodiment of the present invention.

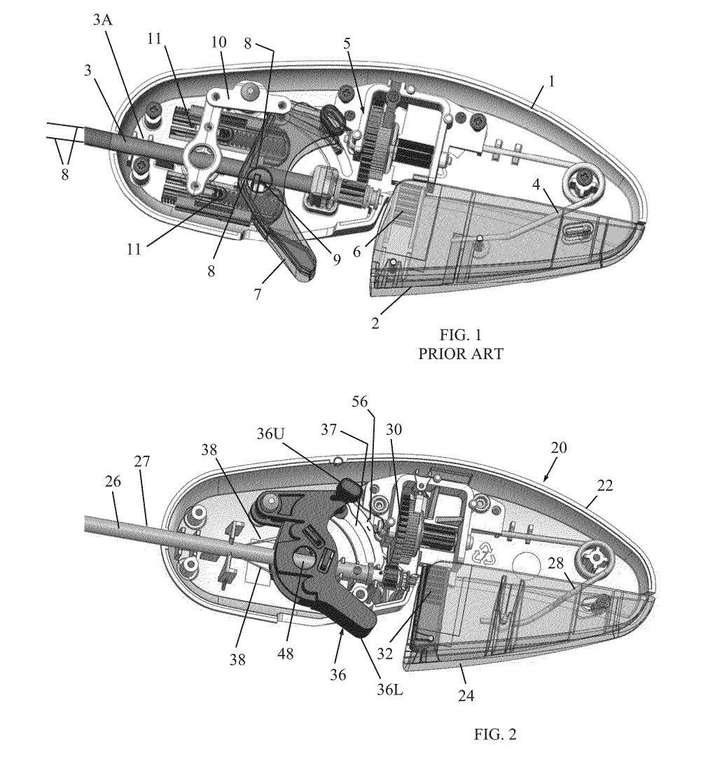

### DETAILED DESCRIPTION OF EMBODIMENTS

Reference is now made to FIG. 2, which illustrates an articulating mechanism 20, constructed and operative in accordance with a non-limiting embodiment of the present invention.

A handle 22 houses a deployment trigger 24 for rotating a drive shaft 26 housed in an outer tube 27. The deployment trigger 24 may be spring-loaded by a spring 28. Squeezing the deployment trigger 24 (upwards, clockwise in the sense of the drawing) causes rotation of the drive shaft 26 through a gear train 30; the motion of the trigger causes a spur gear 32 to mesh with and turn a series of gears of the gear train 30 to cause rotation of the drive shaft 26. An articulating applicator arm 34 (shown in FIG. 4) is connected to the drive shaft 26. In the case of a tacker, operation of the deployment trigger 24 causes the drive shaft 26 to rotate so as to distally advance rotary tacks from applicator arm 34 for deployment in tissue. Up to this point, the construction is similar to that of the prior art.

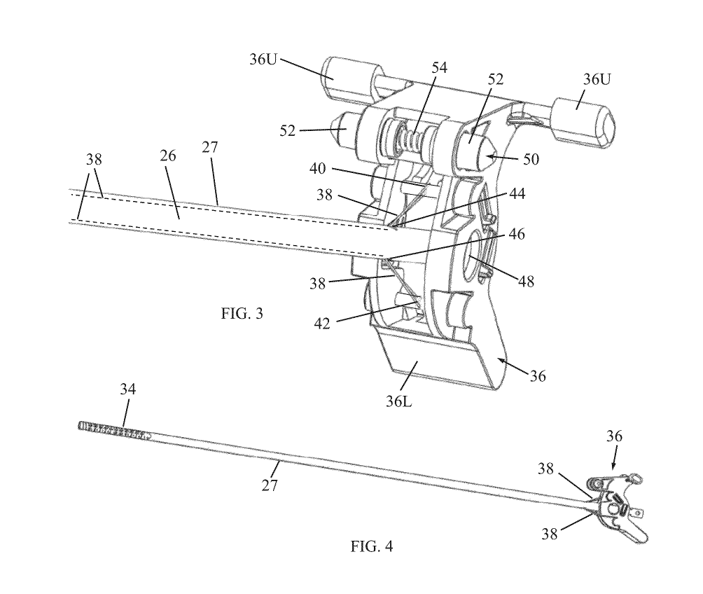

Reference is made additionally to FIG. 3. The articulating mechanism 20 of the illustrated embodiment includes an articulation trigger 36 for articulating the applicator arm 34 at different angles. Superelastic articulating cables 38 are connected to upper and lower points 40 and 42 on articulation trigger 36 and enter drive shaft 26 through entry holes 44 and 46, respectively. The articulating cables 38 run through the length of drive shaft 26 and are connected to the applicator arm 34 (FIG. 4). The superelastic cables 38 may be made of nitinol or any other suitable superelastic material. “Superelasticity” is an elastic reversible property of the material's response to an applied stress. In shape-memory alloys like nitinol, it is caused by a phase transformation between the austenitic and martensitic phases of the crystalline structure of the alloy. In nitinol, up to about 13% (without limitation) deformation strain can be sustained and the material can recover its original shape after removing the stress.

In other embodiments, only one superelastic articulating cable 38 is employed. In other embodiments, more than one articulating cable is employed, but not all of them are superelastic (one could be made of stainless steel, for example).

The articulation trigger 36 may include a lower portion 36L and/or upper portions 36U for easy ambidextrous operation by the surgeon. (As seen in FIG. 2, the upper portions 36U may move in a curved track 37 formed in handle 22.) The articulation trigger 36 pivots about a pivot 48. Pulling the lower part of the articulation trigger 36 towards the handle 22 (upwards, counterclockwise in the sense of FIG. 2) puts tension on (that is, pulls) the lower of the articulating cables 38 and causes the applicator arm 34 (FIG. 4) to articulate downwards. Conversely, pushing the lower part of the articulation trigger 36 away from the handle 22 (downwards, clockwise in the sense of FIG. 2) puts tension on (that is, pulls) the upper of the articulating cables 38 and causes the applicator arm 34 (FIG. 4) to articulate upwards.

The articulation trigger 36 may include a locking mechanism 50 for locking the trigger at a selected operating position. As seen in FIG. 3, locking mechanism 50 includes one or more lugs 52 biased by a biasing device 54 (such as a coil spring 54). In the illustrated embodiment of FIG. 3, there are a pair of lugs 52 urged outwards by biasing device 54 sandwiched between lugs 52. The lugs 52 slide against the inner surface of the handle 22 during operation of trigger 36. One or more dimples or depressions 56 (seen in broken lines in FIG. 2) may be formed in the inner surface of the handle 22. When the lug 52 reaches the depression 56, the biasing device 54 urges the lug 52 to be seated and caught in depression 56, thereby locking the trigger 36 at this position. The surgeon can push lug 52 out of depression 56 so that trigger 36 can be moved to another position or return to its original position. Lug 52 can be pushed out of depression 56 because lug 52 is chamfered or rounded at its end.

The superelasticity of the cables 38 biases the articulation trigger 36 so that trigger 36 will always align with the locking position. This is an elegant solution to the problem of the prior art mentioned above, which requires coil springs and a four-bar linkage mechanism. Instead, in the present invention, no such springs or linkage mechanism are used. The superelastic cables 38 are sufficient to ensure the articulation trigger 36 aligns with the locking position with no backlash or overshoot. The superelasticity of the cable 38 provides tension in the cable 38. The tension in the cables 38 takes the place of coil springs of the prior art and ensures the trigger 36 aligns with the locking position.

What is claimed is:

1. A surgical device comprising:
a handle;
a drive shaft that protrudes from said handle;
an articulating applicator arm connected to said drive shaft; and
an articulation trigger comprising at least one superelastic articulating cable that has one end connected to said articulation trigger and an opposite end connected to said articulating applicator arm.

2. The surgical device according to claim 1, wherein said at least one superelastic articulating cable comprises upper and lower superelastic articulating cables connected to upper and lower points on said articulation trigger and which enter said drive shaft through entry holes.

3. The surgical device according to claim 1, wherein said articulation trigger pivots about a pivot.

4. The surgical device according to claim 1, wherein said articulation trigger comprises a lower portion or upper portions.

5. The surgical device according to claim 4, wherein said upper portions are arranged to move in a curved track formed in said handle.

6. The surgical device according to claim 1, wherein said articulation trigger comprises a locking mechanism for locking said articulation trigger at a selected operating position.

7. The surgical device according to claim 6, wherein said locking mechanism comprises one or more lugs biased by a biasing device, said one or more lugs being arranged to be urged by biasing device into one or more depressions formed in said handle.

8. The surgical device according to claim 1, wherein said handle further comprises a deployment trigger for rotating said drive shaft.

9. A method for using the surgical device of claim 1, comprising operating said articulation trigger so as to cause articulation of said articulating applicator arm.
